(19) United States
(12) Patent Application Publication
(10) Pub. No.: US 2018/0184981 A1
(43) Pub. Date: Jul. 5, 2018

(54) INTRAVASCULAR DEVICES SYSTEMS AND METHODS WITH A SOLID CORE PROXIMAL SECTION AND A SLOTTED TUBULAR DISTAL SECTION

(71) Applicant: KONINKLIJKE PHILIPS N.V., EINDHOVEN (NL)

(72) Inventor: Mark RICHARDSON, San Diego, CA (US)

(21) Appl. No.: 15/741,263

(22) PCT Filed: Jun. 30, 2016
(86) PCT No.: PCT/EP2016/065291

§ 371 Date: Dec. 31, 2017

Related U.S. Application Data
(60) Provisional application No. 62/187,021, filed on Jun. 30, 2015.

(51) Int. Cl. A61B 5/00; A61B 5/0215; A61B 5/026; A61M 25/09

(52) U.S. Cl. CPC A61B 5/6851; A61B 5/0215; A61B 5/026; A61M 2025/09175; A61M 2025/09108; A61M 2025/09083; A61M 25/09

(57) ABSTRACT

The present disclosure is directed to intravascular devices, systems, and methods having a solid core proximal section and a slotted, tubular distal section. In some aspects, a sensing guide wire is provided. The sensing guide wire can include a proximal portion having a solid core member and a plurality of conductors embedded in an outer layer surrounding the solid core member; and a distal portion coupled to the proximal portion, the distal portion having a slotted tubular member and a sensing element, the sensing element being electrically coupled to the plurality of conductors of the proximal portion. In other aspects, methods of forming a sensing guide wire are provided.

### TECHNICAL FIELD

The present disclosure relates to intravascular devices, systems, and methods. In some embodiments, the intravascular devices are guide wires that include a solid core proximal section and a slotted, tubular distal section.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is Fractional Flow Reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular catheters and guide wires are utilized to measure the pressure within the blood vessel, visualize the inner lumen of the blood vessel, and/or otherwise obtain data related to the blood vessel. To date, guide wires containing pressure sensors, imaging elements, and/or other electronic, optical, or electro-optical components have suffered from reduced performance characteristics compared to standard guide wires that do not contain such components. For example, the handling performance of previous guide wires containing electronic components have been hampered, in some instances, by the limited space available for the core wire after accounting for the space needed for the conductors or communication lines of the electronic component(s), the stiffness of the rigid housing containing the electronic component(s), and/or other limitations associated with providing the functionality of the electronic components in the limited space available within a guide wire.

Accordingly, there remains a need for improved intravascular devices, systems, and methods that include a more reliable and consistent connection between two components at a distal tip.

### SUMMARY

The present disclosure is directed to intravascular devices, systems, and methods that include a guide wire having a proximal section formed with a solid core member and a distal section formed with a slotted tubular member.

In some instances, a sensing guide wire is provided that includes a proximal portion having a solid core member and a plurality of conductors embedded in an outer layer surrounding the solid core member; and a distal portion coupled to the proximal portion, the distal portion having a slotted tubular member and a sensing element, the sensing element being electrically coupled to the plurality of conductors of the proximal portion. The distal portion can further include a tip coil coupled to the slotted tubular member. The sensing element can positioned within the slotted tubular member or within a housing. In this regard, the housing can be positioned between the tip coil and the slotted tubular member.

The slotted tubular member can extend to a distal tip of the sensing guide wire. In this regard, an atraumatic tip, such as a solder ball, can be coupled to the distal end of the slotted tubular member to define a distal tip of the guide wire. The plurality of conductors can include between two conductors and six conductors in some implementations. The sensing element can include a pressure sensor, a flow sensor, and/or combinations thereof.

In some instances, a method of assembling a sensing guide wire is provided that includes obtaining a proximal portion having a solid core member and a plurality of conductors embedded in an outer layer surrounding the solid core member; and coupling a distal portion to the proximal portion such that a slotted tubular member of the distal portion extends distally from the proximal portion and a sensing element of the distal portion is electrically coupled to the plurality of conductors of the proximal portion.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic side view of an intravascular device according to an embodiment of the present disclosure.

FIG. 2 is a diagrammatic, schematic side view of a distal portion of the intravascular device of FIG. 1 according to an embodiment of the present disclosure.

FIG. 3 is a cross-sectional side view of the distal portion of the intravascular device of FIGS. 1 and 2 taken along section line 3-3 of FIG. 2 according to an embodiment of the present disclosure.

FIG. 4 is a diagrammatic, schematic side view of a distal portion of the intravascular device of FIG. 1 according to an embodiment of the present disclosure.

FIG. 5 is a diagrammatic, schematic side view of a distal portion of the intravascular device of FIG. 1 according to an embodiment of the present disclosure.

FIG. 6a is a diagrammatic, schematic side view of a proximal portion of the intravascular device of FIG. 1 according to an embodiment of the present disclosure.

FIG. 6b is a cross-sectional end view of the proximal portion of the intravascular device of FIG. 6a according to an embodiment of the present disclosure.

FIG. 6c is a cross-sectional end view of the proximal portion of the intravascular device of FIG. 6a according to an embodiment of the present disclosure.

FIG. 7 is a diagrammatic, schematic side view of a proximal portion of the intravascular device of FIGS. 6a and 6b showing an exposed portion of an embedded conductor according to an embodiment of the present disclosure.

FIG. 8 is a diagrammatic, schematic side view of a proximal portion of the intravascular device of FIGS. 6a, 6b, and 7 showing a conductive band formed over the exposed portion of the embedded conductor of FIG. 7 according to an embodiment of the present disclosure.

FIG. 9 is a diagrammatic, schematic side view of a proximal portion of the intravascular device of FIGS. 6a, 6b, 7, and 8 showing exemplary conductive band configurations according to the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guide wires and catheters. In that regard, catheters may or may not include a lumen extending along its length for receiving and/or guiding other instruments. If the catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a flow sensor, a temperature sensor, an imaging element, an optical fiber, an ultrasound transducer, a reflector, a mirror, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the flexible elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the flexible elongate member, such as a guide wire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm), approximately 0.018″ (0.4572 mm), and approximately 0.035″ (0.889 mm). As such, the flexible elongate members incorporating the electronic, optical, and/or electro-optical component(s) of the present application are suitable for use in a wide variety of lumens within a human patient besides those that are part of or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

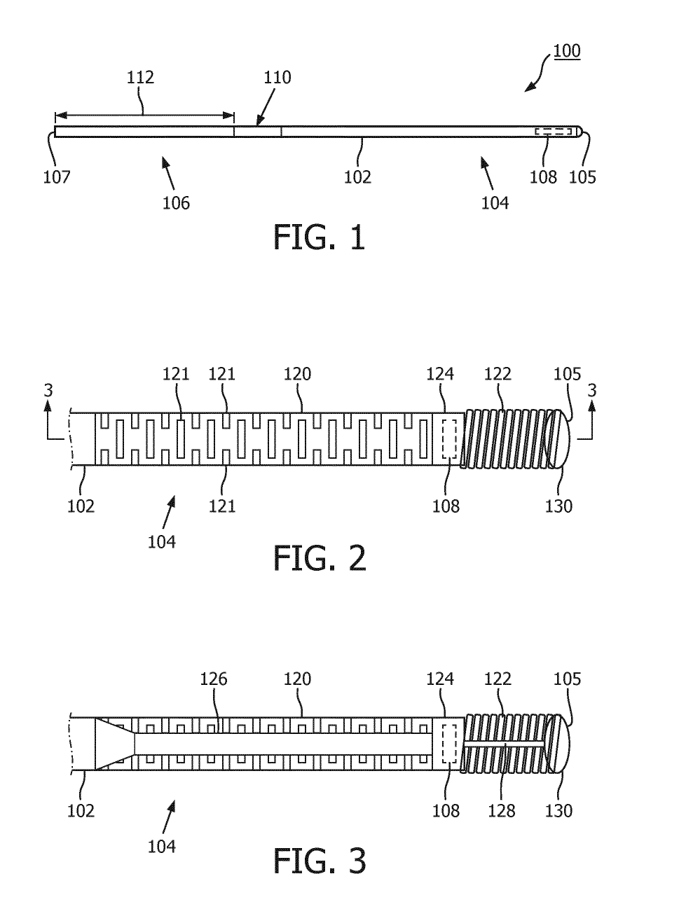

Referring now to FIG. 1, shown therein is a diagrammatic, schematic side view of an intravascular device 100 according to an embodiment of the present disclosure. In that regard, the intravascular device 100 includes a flexible elongate member 102 having a distal portion 104 adjacent a distal tip 105 and a proximal portion 106 adjacent a proximal end 107. A component 108 is positioned within the distal portion 104 of the flexible elongate member 102 proximal of the distal tip 105. Generally, the component 108 is representative of one or more electronic, optical, or electro-optical components. In that regard, the component 108 is a pressure sensor, a flow sensor, a temperature sensor, an imaging element, an optical fiber, an ultrasound transducer, a reflector, a mirror, a prism, an ablation element, an RF electrode, a conductor, and/or combinations thereof. The specific type of component or combination of components can be selected based on an intended use of the intravascular device. In some instances, the component 108 is positioned less than 10 cm, less than 5 cm, or less than 3 cm from the distal tip 105. In some instances, the component 108 is positioned within a housing of the flexible elongate member 102. In that regard, the housing is a separate component secured to the flexible elongate member 102 in some instances. In other instances, the housing is integrally formed as a part of the flexible elongate member 102.

The intravascular device 100 also includes a connector 110 adjacent the proximal portion 106 of the device. In that regard, the connector 110 is spaced from the proximal end 107 of the flexible elongate member 102 by a distance 112. Generally, the distance 112 is between 0% and 50% of the total length of the flexible elongate member 102. While the total length of the flexible elongate member 102 can be any length, in some embodiments the total length is between about 1300 mm and about 4000 mm, with some specific embodiments having a length of 1400 mm, 1900 mm, and 3000 mm. Accordingly, in some instances the connector 110 is positioned at the proximal end 107. In other instances, the connector 110 is spaced from the proximal end 107. For example, in some instances the connector 110 is spaced from the proximal end 107 between about 0 mm and about 1400 mm. In some specific embodiments, the connector 110 is spaced from the proximal end by a distance of 0 mm, 300 mm, and 1400 mm.

The connector 110 is configured to facilitate communication between the intravascular device 100 and another device. More specifically, in some embodiments the connector 110 is configured to facilitate communication of data obtained by the component 108 to another device, such as a computing device or processor. Accordingly, in some embodiments the connector 110 is an electrical connector. In such instances, the connector 110 provides an electrical connection to one or more electrical conductors that extend along the length of the flexible elongate member 102 and are electrically coupled to the component 108. In some embodiments the electrical conductors are embedded within a core of the flexible elongate member 102. In other embodiments, the connector 110 is an optical connector. In such instances, the connector 110 provides an optical connection to one or more optical communication pathways (e.g., fiber optic cable) that extend along the length of the flexible elongate member 102 and are optically coupled to the component 108. Similarly, in some embodiments the optical fibers are embedded within a core of the flexible elongate member 102. Further, in some embodiments the connector 110 provides both electrical and optical connections to both electrical conductor(s) and optical communication pathway(s) coupled to the component 108. In that regard, it should be noted that component 108 is comprised of a plurality of elements in some instances. The connector 110 is configured to provide a physical connection to another device, either directly or indirectly. In some instances, the connector 110 is configured to facilitate wireless communication between the intravascular device 100 and another device. Generally, any current or future developed wireless protocol(s) may be utilized. In yet other instances, the connector 110 facilitates both physical and wireless connection to another device.

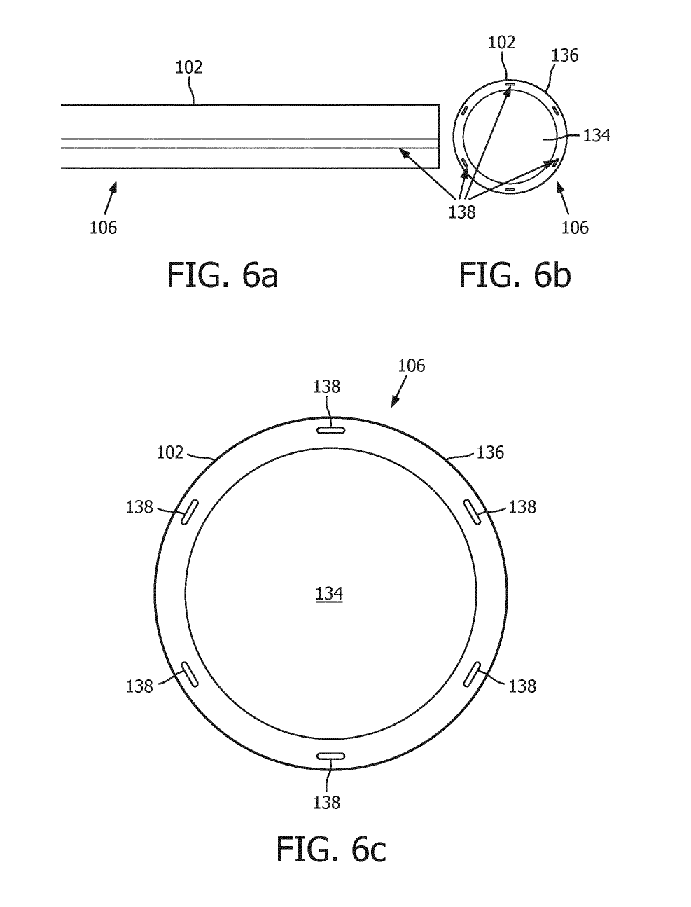

As noted above, in some instances the connector 110 provides a connection between the component 108 of the intravascular device 100 and an external device. Accordingly, in some embodiments one or more electrical conductors, one or more optical pathways, and/or combinations thereof extend along the length of the flexible elongate member 102 between the connector 110 and the component 108 to facilitate communication between the connector 110 and the component 108. In some instances, at least one of the electrical conductors and/or optical pathways is embedded within one or more polymer layers surrounding a core member, as described below with respect to FIGS. 6a-10 and as described in U.S. patent application Ser. No. 14/143,304, filed Dec. 30, 2013, which is hereby incorporated by reference in its entirety. Further, in some instances at least one of the electrical conductors and/or optical pathways is embedded within the core of the flexible elongate member 102, as described in U.S. patent application Ser. No. 14/611,921, filed Feb. 2, 2015, which is hereby incorporated by reference in its entirety. Generally, any number of electrical conductors, optical pathways, and/or combinations thereof can extend along the length of the flexible elongate member 102 between the connector 110 and the component 108, embedded in the core or not. In some instances, between one and ten electrical conductors and/or optical pathways extend along the length of the flexible elongate member 102 between the connector 110 and the component 108. The number of communication pathways and the number of electrical conductors and optical pathways extending along the length of the flexible elongate member 102 is determined by the desired functionality of the component 108 and the corresponding elements that define the component 108 to provide such functionality.

Referring now to FIG. 2, shown therein is a diagrammatic, schematic side view of the distal portion 104 of the intravascular device 100 according to an embodiment of the present disclosure. As shown, the distal portion 104 includes a proximal flexible element 120 and a distal flexible element 122 on each side of a housing 124 containing component 108. The proximal and distal flexible elements 120, 122 can be any suitable flexible element, including slotted tubes, coils, and/or coil-embedded tubes. In the illustrated embodiment of FIG. 2, the proximal flexible element 120 is a slotted tubular member and the distal flexible element 122 is a coil. In this regard, the slotted tubular member defining proximal flexible element 120 includes a plurality of slots 121. Generally, the slots 121 can take on any size(s), shape(s), orientation(s), and/or spacing(s). The particular size(s), shape(s), orientation(s), and/or spacing(s) of the slots 121 can be selected to achieve a desired flexibility and/or transition in flexibilities along the length of the tubular member 120. For example, in some implementations the flexibility of the intravascular device 100 increases as the device extends distally. Accordingly, the size, shape, orientation, and/or spacing of the slots 121 can vary accordingly along the length of the tubular member 120 to achieve the desired change in flexibility. In the illustrated embodiment of FIG. 2, each of the slots 121 has a similar elongated profile extending around a circumference of the tubular member. Further, the spacings between the slots 121 is constant along the length of the tubular member in the illustrated embodiment of FIG. 2. However, it is understood that the slots 121 may take on any size (including height, length, width, depth, etc.), any shape (including geometrical, non-geometrical, and combinations thereof), any orientation (including linear, perpendicular, oblique, and combinations thereof), and/or any spacing (including fixed, variable, symmetric, non-symmetric, and/or combinations thereof) without departing from the scope of the present disclosure.

Referring now to FIG. 3, in some implementations one or more core members extend through at least one of the flexible element 120 or the flexible element 122. For example, as shown in FIG. 3, a core member 126 extends through the proximal flexible element 120. Similarly, a core member 128 extends through the distal flexible element 122. In some implementations, the core members 126 and 128 are an integral component (i.e., the core member 126 extends through the housing 124 to define core member 128). In some instances, the core member 128 is coupled to a shaping ribbon. Generally, the distal flexible element 122, the core members 126, 128, and/or the shaping ribbon are sized, shaped, and/or formed out of particular material(s) to create a desired mechanical performance and/or flexibility for the distal portion 104 of the intravascular device 100. For example, the distal flexible element 122, the core members 126, 128, and/or the shaping ribbon can be formed from metals or metal alloys, such as nickel titanium or nitinol, nickel titanium cobalt, stainless steel, and/or various stainless steel alloys, and/or polymers, such as polyimide, polyethylene, etc. However, any combination of materials can be used in accordance with the present disclosure. Further, in some implementations at least one of the flexible element 120 and/or flexible element 122 does not have a core member or shaping ribbon extending therethrough. For example, in some implementations the flexible element 120 provides suitable structural integrity for the distal portion 104 of the intravascular device 100 without the need for a core member.

A solder ball 130 or other suitable element can be secured to the distal end of the distal flexible element 122. As shown, the solder ball 130 defines the distal tip 105 of the intravascular device 100 with an atraumatic tip suitable for advancement through patient vessels, such as vasculature. In some embodiments, a flow sensor is positioned at the distal tip 105 instead of the solder ball 130.

The distal portion 104 of the intravascular device 100—as well as the proximal portion 106 and the flexible elongate member 102—may be formed using any suitable approach so long as the proximal portion 106 includes a solid core and the distal flexible element 122 includes a slotted tubular member in accordance with the present disclosure. Accordingly, in some implementations the intravascular device 100 includes features similar to the distal, intermediate, and/or proximal sections described in one or more of U.S. Pat. No. 5,125,137, U.S. Pat. No. 5,873,835, U.S. Pat. No. 6,106,476, U.S. Pat. No. 6,551,250, U.S. patent application Ser. No. 13/931,052, filed Jun. 28, 2013, U.S. patent application Ser. No. 14/135,117, filed Dec. 19, 2013, U.S. patent application Ser. No. 14/137,364, filed Dec. 20, 2013, U.S. patent application Ser. No. 14/139,543, filed Dec. 23, 2013, U.S. patent application Ser. No. 14/143,304, filed Dec. 30, 2013, and U.S. patent application Ser. No. 14/611,921, filed Feb. 2, 2015, each of which is hereby incorporated by reference in its entirety.

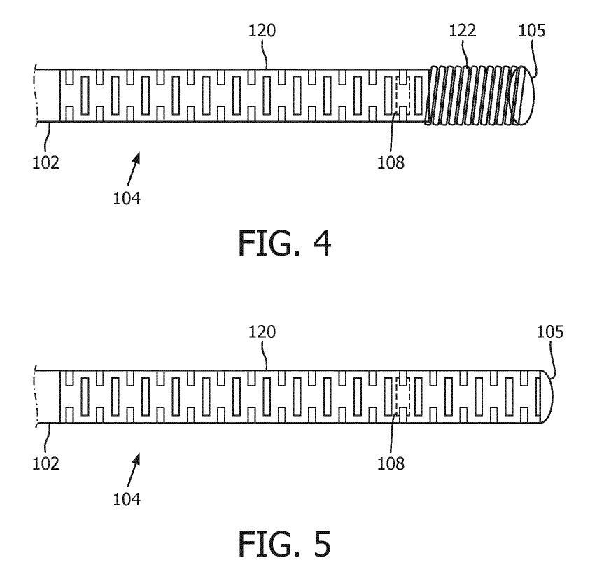

Referring now to FIG. 4, shown therein is a diagrammatic, schematic side view of a distal portion 104 of the intravascular device 100 according to another embodiment of the present disclosure. In particular, the embodiment of FIG. 4 does not a include a separate housing for the component 108. Instead, the component 108 is mounted within the proximal flexible element 120. In this regard, the component 108 may be secured within proximal flexible element 120 using suitable mechanical fastener(s)/coupler(s), adhesive(s), and/or combinations thereof. In some instances, the slots 121 of the tubular member 120 provide fluid access to ambient surroundings for the component 108. For example, in some implementations the slots 121 adjacent to the component 108 are sized, shaped, oriented, and/or spaced to facilitate pressure and/or flow measurements by the component 108. In some instances, eliminating the housing for the component 108 improves the flexibility and/or the consistency of the flexibility of the intravascular device 100 within the distal portion 104, which can improve handling of the intravascular device 100.

Referring now to FIG. 5, shown therein is a diagrammatic, schematic side view of a distal portion 104 of the intravascular device 100 according to another embodiment of the present disclosure. In particular, the embodiment of FIG. 5 does not a include a separate housing for the component 108 or a separate distal flexible element 122. Instead, the component 108 is mounted within the proximal flexible element 120 that extends to the distal tip 105 of the intravascular device 100. In this regard, the component 108 may be secured within proximal flexible element 120 using suitable mechanical fastener(s)/coupler(s), adhesive(s), and/or combinations thereof. In some instances, the slots 121 of the tubular member 120 provide fluid access to ambient surroundings for the component 108. For example, in some implementations the slots 121 adjacent to the component 108 are sized, shaped, oriented, and/or spaced to facilitate pressure and/or flow measurements by the component 108. Further, in some instances the slots 121 of the tubular member 120 vary along the length of the intravascular device to provide increased flexibility adjacent the distal tip 105. In particular, the slots 121 adjacent the distal tip 105 can be sized, shaped, oriented, and/or spaced to provide similar flexibility to a tip coil, but using a single component. In the illustrated embodiment, the slots 121 adjacent the distal tip 105 have a reduced spacing relative to the slots 121 positioned more proximally (e.g., proximal of the component 108). In some instances, eliminating the housing for the component 108 and/or the separate distal flexible element 122 improves the manufacturability of the device by eliminating the need to couple multiple discrete components together, which also eliminates potential areas for device failure either during manufacture or use. Further, by using a single, integral flexible element along the distal portion 104 of the intravascular device, the flexibility and/or the consistency of the flexibility of the intravascular device 100 can be precisely controlled by selecting the size(s), shape(s), orientation(s), and/or spacing(s) of the slots 121 to achieve a desired flexibility and/or handling profile.

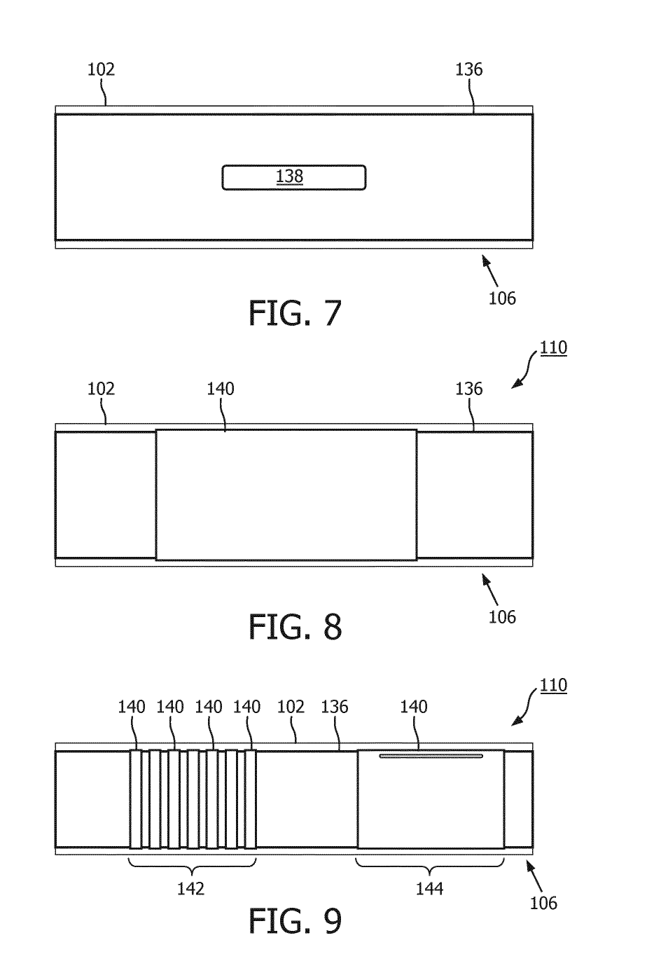

Referring now to FIGS. 6a and 6b, shown therein are aspects of a proximal portion 106 of the intravascular device 100 according to an embodiment of the present disclosure. In particular, FIG. 6a is a diagrammatic, schematic side view of the proximal portion 106, while FIG. 6b is a cross-sectional end view of the proximal portion 106 according to an embodiment of the present disclosure. As shown, the flexible elongate member 102 of the proximal portion 106 includes a core member 134 surrounded by an outer layer 136 with conductors 138 impregnated therein. The core member 134 can be formed of a suitable material such as stainless steel, nickel and titanium alloy (Nitinol), polyetheretherketone, heat straightened 304 stainless steel, or other metallic or polymeric materials. The outer layer 136 can be formed of a suitable polymeric material. In that regard, the outer layer 136 can coated onto the core member 134 using wire coating techniques. As the thickness of the coating is built up, conductors 138 can be introduced into the coating process such that they become coated by and embedded within the outer layer 136. The outer layer 136 may utilize any polymeric material, but in some instances includes polyimide. The conductors 138 can be spaced about the circumference of the core member 134 in any suitable manner, including symmetric and non-symmetric patterns. In certain embodiments, the conductors 138 are spaced substantially equally around a circumference of the core member 134 as shown in FIGS. 6b and 7.

As noted above, generally any number of conductors can be utilized. For example, in some implementations with an electrical, solid-state sensor (e.g., pressure, temperature, flow, etc.) two conductors are utilized to connect to the sensor. In other implementations utilizing a piezo-resistive sensor (e.g., pressure sensor) three conductors are utilized. In yet other implementations utilizing ultrasound sensor(s) for imaging, four conductors are utilized (e.g., as described in U.S. Pat. No. 8,864,674 titled “CIRCUIT ARCHITECTURES AND ELECTRICAL INTERFACES FOR ROTATIONAL INTRAVASCULAR ULTRASOUND (IVUS) DEVICES” and/or U.S. Patent Application Publication No. 2014/0187960 titled “INTRAVASCULAR ULTRASOUND IMAGING APPARATUS, INTERFACE ARCHITECTURE, AND METHOD OF MANUFACTURING,” each which is hereby incorporated by reference in its entirety). FIG. 6b illustrates an embodiment utilizing three conductors 138, while FIG. 6c illustrates an embodiment utilizing six conductors 138. In addition, the core 134 may also be utilized as a conductor in some embodiments. The number of conductors 138 utilized can be selected based on the number and/or type(s) of sensing components utilized.

In certain embodiments, after reaching a desired outer diameter for the outer layer 136, a final coating can be applied to the proximal portion 106 and the intravascular device 100. Any suitable material that can provide lubricity may be used, including hydrophilic and hydrophobic coatings. Exemplary coating materials include PTFE impregnated polyimide, silicone-based coatings, hydrophilic coatings, and hydrophobic coatings.

Referring now to FIG. 7, shown therein is a diagrammatic, schematic side view of the proximal portion 106 of the intravascular device 100 showing an exposed portion of a conductor 138 embedded in the outer layer 136 according to an embodiment of the present disclosure. As shown, one or more sections of the outer layer 136 are modified to expose corresponding sections of the embedded conductor(s) 138. Any suitable technique may be used to expose the sections of conductor(s) 138, including chemical etching, mechanical cutting and shearing, laser ablation, and/or combinations thereof. In certain embodiments, laser ablation is used to cut away specific sections of the outer layer 136 (e.g., having a particular size, shape, depth, etc.) to expose the embedded conductor(s) 138. Circumferential ablation may be utilized in some instances. Laser ablation of polymeric material is known in the art and can be accomplished by known techniques, such as those described in Kumagai (Applied Physics Letters, 65(14):1850-1852, 2004); Sutcliffe (Journal of Applied Physics, 60(9):3315-3322, 1986), and Blanchet et al. (Science, 262(5134):719-721, 1993), each of which is incorporated by reference herein in its entirety.

In some instances, a reference ring at a proximal and/or distal end of the flexible elongate member 102 may be ablated to identify where the conductors 138 reside in the outer layer 136 with respect to the circumference of the device. In that regard, the distal end of the conductive wires may be ground to the specified grind profile for coupling to the component 108, either directly or indirectly. For example, the conductors 138 may be coupled to the component 108 using soldering welding, one or more additional conductive members, leads, and/or other known techniques. In some instances, sections of the outer layer 136 are removed to expose the distal portions of the conductors 138 that will be coupled to the component 108. via additional conductors. In that regard, in some instances the distal end of flexible elongate member 102 is coupled to a distal portion similar to those described above with respect to FIGS. 2-5. In such instances, the conductors 138 may be coupled to conductors that extend along and/or through the proximal flexible element 120 to the component 108. Further, the flexible elongate member 102 may be coupled to the proximal flexible element, directly or indirectly, using mechanical coupling(s), adhesive(s), solder(s), weld(s), and/or combinations thereof. In some instances, the flexible elongate member 102 is coupled to a distal section, intermediate section, and/or proximal section similar to those described in one or more of U.S. Pat. No. 5,125,137, U.S. Pat. No. 5,873,835, U.S. Pat. No. 6,106,476, U.S. Pat. No. 6,551,250, U.S. patent application Ser. No. 13/931,052, filed Jun. 28, 2013, U.S. patent application Ser. No. 14/135,117, filed Dec. 19, 2013, U.S. patent application Ser. No. 14/137,364, filed Dec. 20, 2013, U.S. patent application Ser. No. 14/139,543, filed Dec. 23, 2013, U.S. patent application Ser. No. 14/143,304, filed Dec. 30, 2013, and U.S. patent application Ser. No. 14/611,921, filed Feb. 2, 2015, each of which is hereby incorporated by reference in its entirety.

Referring now to FIG. 8, shown therein is a diagrammatic, schematic side view of the proximal portion 106 of the intravascular device 100 showing a conductive band 140 formed over the exposed portion of the embedded conductor of FIG. 7 according to an embodiment of the present disclosure. As shown, a conductive material can be applied to the flexible elongate member 102 over the exposed sections of the conductors 138. The conductive material covers the exposed sections of conductors 138 to define a conductive band 140 that is in contact with an exposed conductor 138. Typically, each conductive band 140 will be connected to a single conductor 138 (at one or more locations along the length of the conductor) such that the conductive band 140 serves as an external electrical connector for that conductor 138. However, in some implementations a conductive band 140 may be connected to two or more conductors 138. The conductive material will generally be a metal, such as gold. Generally, any suitable technique can be used to apply the conductive material to the exposed conductors. In certain embodiments, the conductive material is printed and sintered onto the exposed sections of conductive wires. Printing and sintering of metal is known. See, for example, Kydd (U.S. Pat. Nos. 5,882,722 and 6,036,889), Karapatis et al. (Rapid Prototyping Journal, 4(2):77-89, 1998), and Kruth et al., (Assembly Automation, 23(4):357-371, 2003), the content of each of which is incorporated by reference herein in its entirety.

Any desired pattern of conductive material may be placed onto the flexible elongate member 102 to define the conductive band 140. For example, the conductive band can be solid, multiple rings, a spiral, or any other pattern that provides the optimum functionality. To that end, FIG. 9 shows two exemplary conductive band configurations. In particular, the arrangement on the left hand side of the drawing illustrates a plurality of conductive bands 140 each connected to a common conductor 138 to define a connector 142, while the configuration on the right shows a solid conductive band 140 that defines a connector 144 for another conductor 138 of the flexible elongate member. The connectors 142 and 144 can be part of a connector 110 as described above with respect to FIG. 1.

Guide wires of the present disclosure can be connected to an instrument, such as a computing device (e.g., a laptop, desktop, or tablet computer) or a physiology monitor, that converts the signals received by the sensors into pressure and velocity readings. The instrument can further calculate Coronary Flow Reserve (CFR) and Fractional Flow Reserve (FFR) and provide the readings and calculations to a user via a user interface. In some embodiments, a user interacts with a visual interface to view images associated with the data obtained by the intravascular devices of the present disclosure. Input from a user (e.g., parameters or a selection) are received by a processor in an electronic device. The selection can be rendered into a visible display.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the features of various embodiments can be combined with features of different embodiments. One or more steps can be added to or removed from the methods described herein. A person of ordinary skill in the art will understand that the steps of the method can be performed in an order different than the order described herein. It is understood that such variations may be made to the foregoing without departing from the scope of the present disclosure. Accordingly, it is appropriate that the appended claims be construed broadly and in a manner consistent with the present disclosure.

What is claimed is:

1. A sensing guide wire, comprising:
a proximal portion having a solid core member and a plurality of conductors embedded in an outer layer surrounding the solid core member; and
a distal portion coupled to the proximal portion, the distal portion having a slotted tubular member and a sensing element, the sensing element being electrically coupled to the plurality of conductors of the proximal portion.

2. The guide wire of claim 1, wherein the distal portion further comprises a tip coil coupled to the slotted tubular member.

3. The guide wire of claim 2, wherein the sensing element is positioned within the slotted tubular member.

4. The guide wire of claim 2, further comprising a housing, the sensing element being disposed within the housing.

5. The guide wire of claim 4, wherein the housing is positioned between the tip coil and the slotted tubular member.

6. The guide wire of claim 1, wherein the sensing element is positioned within the slotted tubular member.

7. The guide wire of claim 6, wherein the slotted tubular member extends to a distal tip of the sensing guide wire.

8. The guide wire of claim 1, wherein the plurality of conductors includes between two conductors and six conductors.

9. The guide wire of claim 1, wherein the sensing element comprises a pressure sensor or flow sensor.

10. A method of assembling a sensing guide wire, the method comprising:
obtaining a proximal portion having a solid core member and a plurality of conductors embedded in an outer layer surrounding the solid core member; and
coupling a distal portion to the proximal portion such that a slotted tubular member of the distal portion extends distally from the proximal portion and a sensing element of the distal portion is electrically coupled to the plurality of conductors of the proximal portion.

11. The method of claim 10, further comprising coupling a tip coil to the slotted tubular member.

12. The method of claim 11, further comprising positioning and securing the sensing element within the slotted tubular member.

13. The method of claim 11, further comprising coupling a housing to the slotted tubular member and securing the sensing element within the housing.

14. The method of claim 13, wherein coupling the housing to the slotted tubular member includes positioning the housing between the tip coil and the slotted tubular member.

15. The method of claim 10, further comprising positioning and securing the sensing element within the slotted tubular member and coupling an atraumatic tip to a distal end of the slotted tubular member.

16-20. (canceled)
